(19) United States
(12) Patent Application Publication
(10) Pub. No.: US 2009/0069902 A1
(43) Pub. Date: Mar. 12, 2009

(54) ARTIFICIAL CONTRACTILE TISSUE

(75) Inventors: Piergiorgio Tozzi, Lausanne (CH); Daniel Hayoz, Villars-sur-Glane (CH); Ludwig Von Segesser, Lausanne (CH)

Correspondence Address: YOUNG & THOMPSON, 209 Madison Street, Suite 500, ALEXANDRIA, VA, 22314, US

(73) Assignee: Nanopowers S.A., Lausanne (CH)

(21) Appl. No.: 12/159,783

(22) PCT Filed: Dec. 28, 2006
(86) PCT No.: PCT/IB06/55044

§ 371 Date: Sep. 25, 2008

(30) Foreign Application Priority Data
Jan. 4, 2006 (IB) PCT/IB2006/050033

(51) Int. Cl. A61F 2/02; A61F 2/08

(52) U.S. Cl. 623/23.72; 623/13.11

(57) ABSTRACT

Artificial contractile tissue including a structure (b,f) and several fibers (a,g) of variable length which are fixed at their ends to the structure (b,f). The fibers (a,g) are made of a contractile material which can be activated by an activator in such a way as to provide a tissue in a rest or in an activated position, the rest position being defined with non-rectilinear fibers (a,g) and the activated position being defined with fibers (a,g) of reduced length; the transition from the rest towards the activated position or vice-versa being defined by a fiber movement along a lateral direction which is perpendicular with respect to the fiber length.

### TECHNICAL FIELD

The present invention relates to an artificial contractile tissue generally devised to be used in the medical field. Such a tissue may be advantageously used to assist muscular contraction, in particular atrial contraction of patients with atrial fibrillation.

### BACKGROUND OF THE INVENTION

Artificial supports to assist muscular contraction are disclosed in Japanese patent applications JP 2001112796 and JP 7008515.

The devices described in this prior art act as muscle fibers and are therefore not adapted to completely replace a muscle tissue.

US patent application US 2005/0020871 discloses an artificial beating tissue based on nanotechnology actuators as source of one or more spatially oriented forces which are used to exert an extra pressure on the cardiac region to be assisted. To this effect, a network of contractile elements connected with longitudinal elements is provided. The network is embedded in an elastomeric material. Activation of the contractile elements causes a reduction in their length that is associated to the contraction of the web.

### SUMMARY OF THE INVENTION

The objective of the present invention is to provide an improved artificial contractile tissue.

This objective has been reached according to the present invention by an artificial contractile tissue comprising a structure and several fibers of variable length which are fixed at their ends to the structure. The fibers are made of a contractile material which can be activated by an activator, e.g. an electric current/voltage, in such a way as to provide a tissue in a rest or in an activated position, the rest position being defined with non-rectilinear fibers and the activated position being defined with fibers of reduced length. The transition from the rest towards the activated position or vice-versa is defined by a fiber movement along a lateral direction which is perpendicular with respect to the fiber length.

In one embodiment, the structure is rigid and forms a closed line, the ends of each fibers being fixed to two separate points of the structure.

The closed line may be comprised in a plane and may form any shape, regular or not, for instance a circle, an ellipse, a square or a triangle.

In a preferred embodiment, the structure has an annular shape and each fiber forms a diameter of the annular structure. This means that all fibers are crossing each other at the center of the annular structure. At this point, the fibers are advantageously glued to each other.

In another embodiment, the structure has an annular shape and each fiber forms a loop around a central piece, called pivot hereafter, which is located at the center of the annular structure.

On one or both sides of the tissue, a membrane, e.g. made of silicone, may cover the fibers.

When using a planar structure, at rest position, the plane preferably forms an angle of 20 to 35° with the fiber ends.

Advantageously the external surface of the structure comprises a sewing surface, for instance a Dacron™ coating.

In another embodiment the structure is a flexible sheet, for instance woven or knitted tissue containing Kevlar™ or carbon fibers. In this case the contractile fibers may be distributed and fixed at their ends to appropriate locations on the sheet.

In a preferred embodiment several protrusions are distributed on the sheet surface, each protrusion being adapted to hold a fiber middle part, in such a way that activation of the fiber results in a lateral movement of the protrusion and therefore a contraction of the sheet.

In another embodiment the contractile fibers are knitted in the flexible sheet, on both sides, in such a way that the flexible sheet itself avoids shortcuts when an electric current is used to activate the contractile fibers. The fiber activation results in a movement of the flexible sheet ends in any desired direction.

If the activator is an electric current an isolating substance preferably covers the fibers. For instance, fibers may be inserted in ePTFE tubes.

Any suitable material can be used for the fibers, in particular Electro Active Polymers (EAP), Electro Active Ceramics (EAC), Shape Memory Alloys (SMA).

SMA undergo changes in shape and hardness when heated or cooled, and do so with great force. The mechanism of the shape memory effect is a diffusionless phase transformation as a solid, in which atoms move cooperatively, often by shear like mechanisms. SMA have a uniform crystal structure that radically changes to a different structure at a specific temperature, When the SMA is below this transition temperature (martensitic state) it can be stretched and deformed without permanent damages. After the SMA has been stretched, if it is heated (i.e. electrically) above its transition temperature (austenite state), the alloy recovers to the un-stretched shape and completely reverses the previous deformation.

Moreover, SMA are capable to lift thousand times their own weight. SMA have the ability to recover from plastic deformation, which is sustained below critical temperature, by heating, and they can work under tension, compression, bending or torsion.

Table 1 below shows a comparison of the properties of materials which may be used for artificial muscles: Electro Active Polymers, Shape Memory Alloys and Electro Active Ceramics.

TABLE 1

| | ElectroActive | Shape Memory | Electroactive |
|---|---|---|---|
| Property | Polymers (EAP) | Alloys (SMA) | Ceramics (EAC) |
| Actuation | >10% | <8% | 0.1-0.3% |
| Displacement | | | |
| Force (Mpa) | 10-30 | 700 | 30-40 |
| Reaction speed | μsec | sec | μsec |
| Density | 1-2.5 g/cc | 5 g/cc | 6-8 g/cc |
| Drive voltage | 4-7 V | 4-50 V | 50-800 V |
| Fracture | resilient, elastic | elastic | fragile |
| toughness | | | |

Even if the energetic efficiency of these materials is lower than conventional electric and magnetic pumps (only 5% of the electricity potential for work becomes a usable physical force with 95% lost as heat), their high strength-to-weight ratio, small size and low operating voltages, allow the development of devices that would be difficult or impossible to make using conventional motors with overall better performance than other systems.

A suitable SMA material for the contractible fibers is Nitinol™. In this case the fibers can be stretched by as much as 4% when below the transition temperature, and when heated, they contract, recovering thereby to their original, shorter length with a usable amount of force in the process. Temperature range is 37-50° C.

Other particularly interesting materials are Biometal fibers (BMF) and Biometal helix (BMX) commercialized by Toki Corporation Inc., Japan. Those materials are able to reversibly contract upon a controlled heating caused by the supply of an electric current/voltage.

### BRIEF DESCRIPTION OF THE DRAWINGS

The invention is discussed below in a more detailed way with examples illustrated by the following figures:

FIG. 1 shows a front view of a first embodiment of the invention.

FIG. 2 shows a side view of the embodiment of FIG. 1.

FIG. 3 shows the embodiment of FIG. 1 in a rest position.

FIG. 4 shows the embodiment of FIG. 1 in an activated position.

FIGS. 5A and 5B show a second embodiment of the invention in which a central pivot avoids contractile fibers crossing each others.

FIGS. 6A and 6B show an enlargement of the pivot of FIG. 5.

FIG. 7 shows a third embodiment of the invention.

FIG. 8 shows a forth embodiment of the invention where the contractile fibers are knitted in a flexible sheet.

FIGS. 9A and 9B show the working principle of the third and forth embodiments.

FIG. 10 shows the tissue of the third and forth embodiment in a rest and in an activated position.

### LIST OF REFERENCES USED IN THE FIGURES

a) fiber
b) annular structure
c) apex
d) membrane
e) sewing surface
f) flexible sheet
g) fiber
h) protrusion
i) groove
j) pivot
k) cap

### DETAILED DESCRIPTION

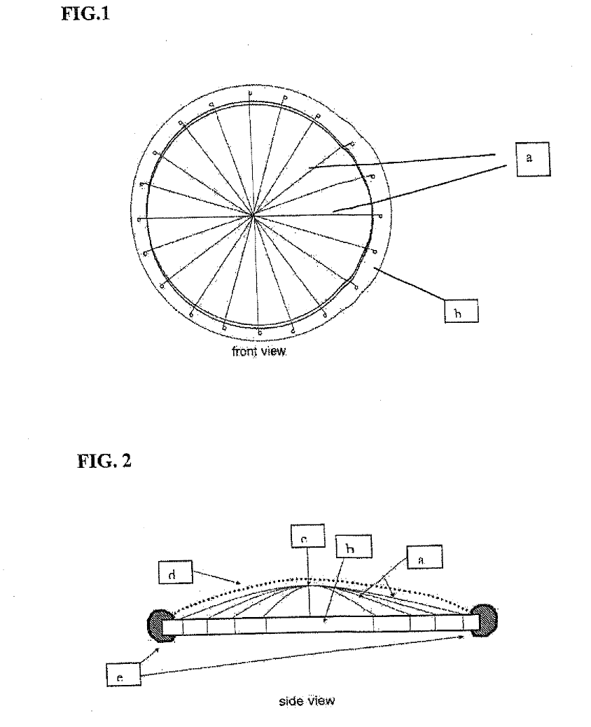

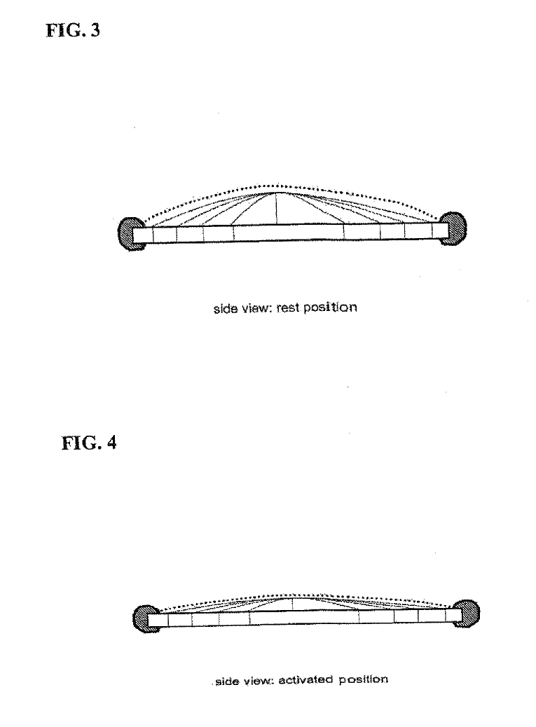

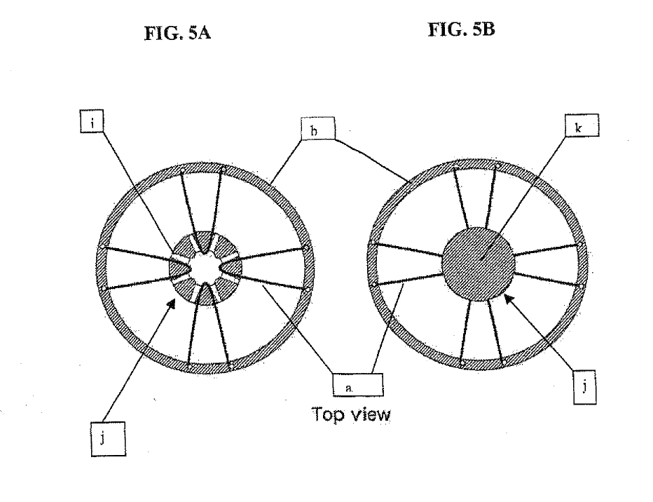

The embodiment illustrated on FIGS. 1 to 4 is defined by a rigid annular structure b. The fibers a are distributed across the ring and pass through the middle point of the structure b in such a way as to create a dome forming an angle of preferably 20 to 35° with respect to the ring plane. The point c where fibers cross each others in the middle point of the ring is the apex of the dome. When an electric current/voltage is applied to the fibers a, their length is reduced and the apex c gets closer to the ring plane of the ring as represented in FIG. 2. When the dome is applied on the surface of the upper chamber of the heart (atrium), its electrically activated movement pushes the wall of the atrium and its content (the blood). The blood is therefore forced to move into the ventricle. This is the mechanical support to the blood circulation.

The ring may be made of plastic, e.g. Delrin™ and may have other shapes than a circular (ellipse, eight shape, etc. . . . ).

Bench tests have demonstrated that a 55 mm dome made of BMX200 can pump 80 ml of water against a pressure of 15 mmHg each time it is activated (contraction). With a rate of contractions of 60 times per minute, a total volume of 480 ml per minute of water may be pumped.

In order to avoid shortcuts, fibers a are isolated, e.g. inserted in ePTFE tubes having an inner diameter which may be of 400 μm. The ePTFE tubes are preferably glued together at the apex c.

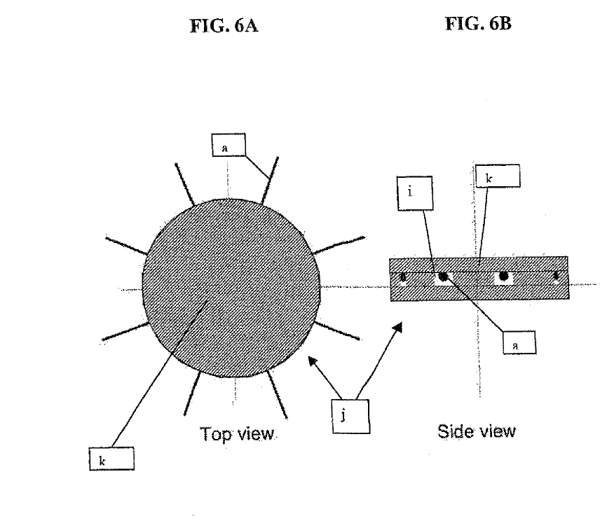

Another mean to avoid shortcuts is to insert a pivot j at the apex c as illustrated in FIGS. 5A to 6B. The pivot j is made of plastic, has a round shape with grooves i on its surface. The fibers a pass into the grooves i forming a loop through the pivot j. The pivot j is furthermore covered by a cap k to ensure proper maintenance of the fibers a in the grooves i.

A thin silicone membrane d, e.g. 100 μm thick, covers the inner and outer part of the dome to provide thermo isolation of the dome thereby reducing the risk of burn lesions on the heart surface.

On the external surface of the ring b, a coating, e.g. made of Dacron™, is fixed to provide a sewing surface e for the connection to the heart.

Advantageously the dome is sutured on the external surface of the upper chamber of the heart (atrium) in the rest position in such a way the atrium completely fills the inner part of the dome.

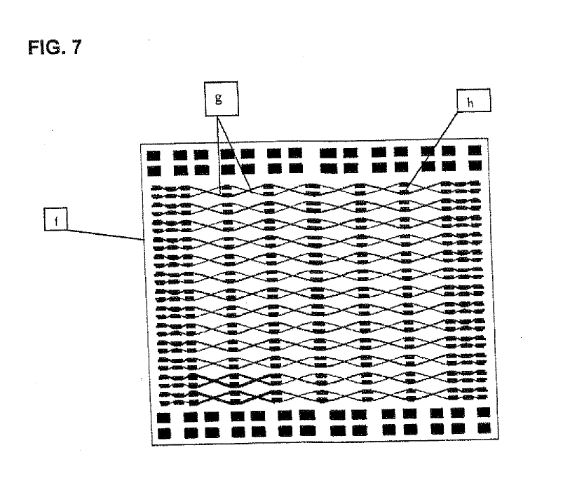

FIGS. 7 to 10 show another embodiment of an artificial contractile tissue according to the invention which comprises a flexible sheet.

It should be pointed out at this stage that in the present invention, “flexible sheet” does not mean “elastomeric material” as disclosed in prior art application US 2005/0020871. A flexible sheet as presently defined can be folded but not extended or contracted.

In this embodiment (see FIG. 7), the artificial muscle essentially consists of a matrix comprising contractible fibers g, e.g. Nitinol™ fibers, and a flexible sheet f made of polyimide. The matrix includes several protrusions h which may be made of copper and which act as pivots. The fibers g pass around the protrusions h in such a way to create a series of waives. At the matrix edges the fiber ends are fixed, e.g. glued, to the protrusions. Fibers cross each other with an angle of about 40°. In the illustrated embodiment, there are 26 lines of fibers having each 7 waives. Protrusions close to matrix's edges are used as electric contacts (positive and negative electrodes).

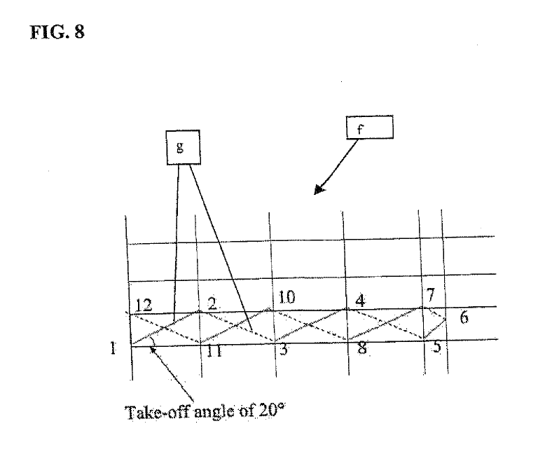

In another embodiment a flexible sheet f is partially and schematically illustrated on FIG. 8. The sheet f is made of polyester tissue which may be reinforced with Kevlar™ or carbon fibers. Preferably Nitinol™ fibers (BMF) g are knitted in the flexible sheet f, on both sides, in such a way that the sheet f itself avoids shortcuts when an electric current is used to activate the contractile fibers. On FIG. 8, only one fiber g is illustrated. The numbering shows the successive locations where the fiber g is crossing the sheet f. A full line represents a fiber portion which is above the sheet f while a dashed line represents a fiber portion which is below the sheet f. The contractile fibers g are knitted in the tissue in such a way to create a series of waves as described in the previous embodiment and following the working principle discussed below. The difference is that in the present embodiment fibers g are on both sides of the flexible sheet f. Waves are therefore present on both sides of the sheet f and the activation of the fibers g results in a movement of the sheet ends in any desired direction.

Several matrix can be joined together in parallel (to increase the pulling force) and/or serial (to increase the length of the displacement) configuration for different clinical applications.

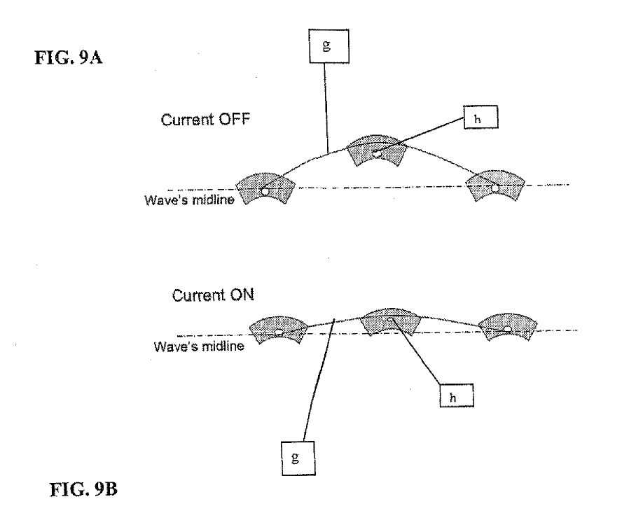

The working principle of the previous cited embodiment will be discussed below and illustrated on FIGS. 9A and 9B.

When electrically activated, the fibers g reach their transitional temperature and may shrink 4% of their length, pulling consequently protrusions h down to the wave's midline. Because protrusions h are fixed to the matrix, fiber's activation results in matrix movement.

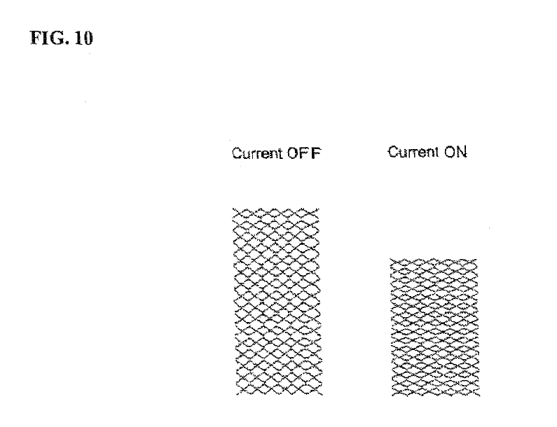

The axe of the movement of the matrix is orthogonal with respect to the fiber movement. Synchronous activation of the 26 fibers causes the matrix shrinking of about 25% as illustrate in FIG. 10.

The matrix discussed here is able to develop about 240 gf over 6 mm displacement which corresponds to 0.1 W.

A Drive Unit (DU) and a Power Source (PS) are necessary to control and power matrix movement.

The DU is basically a microprocessor that distributes current to fibers. Intensity, width and rate of the electrical stimuli are determined according to the application of the matrix.

The PS may be a rechargeable battery.

The present invention has several applications in the medical field, in particular:
Artificial Muscle for cardiac assist. In patients suffering from Chronic Atrial Fibrillation, the contractile function of the upper chambers of the heart (called atria) is lost and cannot be restored by any means. The heart is therefore weaker than normal. For instance two domes can be placed around the upper chambers of the heart (atria) and sutured to the external surface of the heart (epicardium). When simultaneously activated (e.g. 1 Hz frequency) they squeeze the atrium from outside and replace the natural function of this part of the heart. Such a configuration may offer a force of about 500 g and a displacement of about 25 mm, which corresponds to a power of about 1 W.

The drive unit is similar to that currently used for single chamber cardiac pacemakers: it detects ventricular electrical activity thanks to an epicardial electrode and provides control of current direction, intensity and frequency of activation of contractile elements: the contraction can be synchronous, asynchronous, sequential or others in order to have the most appropriate three dimensional deformations to compress atria and achieve the optimal ventricular filling. Lithium-manganese dioxide batteries (500 mA for 3.2V) provide the power supply and can last for 6 h. A percutaneous energy transfer supply can be developed for battery recharge during the night, as routinely done with other ventricular assist devices like LionHeart.
Treatment of congestive heart failure.
Treatment of neuromuscular diseases causing paralysis and post traumatic paralysis of lower and/or upper extremities, to increase muscular strength.
More generally, assisting contraction of an organ (stomach, bladder, urethra, etc.).

1-15. (canceled)

16. Artificial contractile tissue comprising a rigid structure forming a closed line and several fibers of variable length which are fixed at their ends to two separate points of said structure and which are distributed across said structure in such a way to create a dome; said fibers being made of a contractile material which can be activated by an activator in such a way as to provide a tissue in a rest or in an activated position, the rest position being defined with non-rectilinear fibers and the activated position being defined with fibers of reduced length; the transition from the rest towards the activated position or vice-versa being defined by a fiber movement along a lateral direction which is perpendicular with respect to the fiber length, so that the dome gets closer to said structure.

17. Artificial contractile tissue according to claim 16, wherein said closed line is comprised in a plane.

18. Artificial contractile tissue according to claim 16, wherein said structure has an annular shape, each fiber forming a diameter of said annular structure.

19. Artificial contractile tissue according to claim 16, wherein said structure has an annular shape, each fiber passing into a groove of a central pivot and forming a loop across said pivot.

20. Artificial contractile tissue according to claim 16, furthermore comprising a membrane which covers the fibers on one side of the tissue.

21. Artificial contractile tissue according to claim 20, comprising another membrane which covers the other side of the tissue.

22. Artificial contractile tissue according to claim 17, wherein at rest position, said plane forms an angle of 20 to 35° with the fiber ends.

23. Artificial contractile tissue according to claim 16, wherein the external surface of the structure comprises a sewing surface.

24. Artificial contractile tissue according to claim 16, wherein said activator is an electric current/voltage.

25. Artificial contractile tissue according to claim 17, furthermore comprising a membrane which covers the fibers on one side of the tissue.

26. Artificial contractile tissue according to claim 18, furthermore comprising a membrane which covers the fibers on one side of the tissue.

27. Artificial contractile tissue according to claim 19, furthermore comprising a membrane which covers the fibers on one side of the tissue.
